(19) United States
(12) Patent Application Publication
(10) Pub. No.: US 2022/0210298 A1
(43) Pub. Date: Jun. 30, 2022

(54) CAMERA MODULE AND ENDOSCOPE

(71) Applicant: SHENZHEN YATEKS CO., LTD, SHENZHEN (CN)

(72) Inventors: Jiangfan YAN, SHENZHEN (CN); Juhao CHEN, SHENZHEN (CN); Zhongkai WANG, SHENZHEN (CN); Hongquan GUAN, SHENZHEN (CN); Haijuan ZOU, SHENZHEN (CN); Xiang ZHENG, SHENZHEN (CN)

(73) Assignee: SHENZHEN YATEKS CO., LTD

(21) Appl. No.: 17/359,121

(22) Filed: Jun. 25, 2021

(30) Foreign Application Priority Data
Dec. 29, 2020 (CN) 202011590826.X

(51) Int. Cl. H04N 5/225; G03B 17/55; G03B 15/02; A61B 1/06; A61B 1/05

(52) U.S. Cl. CPC H04N 5/2254; G03B 17/55; G03B 15/02; H04N 2005/2255; H04N 5/2256; A61B 1/0607; A61B 1/05; H04N 5/2252

(57) ABSTRACT

The present disclosure relates to the field of optical technologies and provides a camera module and an endoscope. The camera module includes: a connecting base, including a first connecting member and a first circuit board; wherein the first circuit board is installed in the first connecting member; and a camera, including a housing, a second circuit board, a light source assembly, and a lens assembly; wherein the second circuit board, the light source assembly, and the lens assembly are respectively installed in the housing; the housing is removably connected to the first connecting member; the first circuit board and the second circuit board are electrically connected to each other.

### CROSS REFERENCE TO RELATED APPLICATIONS

This application claims foreign priority of Chinese Patent Application No. 202011590826.X, filed on Dec. 29, 2020 in the China National Intellectual Property Administration, the disclosures of all of which are hereby incorporated by reference.

### TECHNICAL FIELD

The present disclosure relates to the field of optical technologies, and in particular to a camera module and an endoscope.

### BACKGROUND

Endoscopes are widely used in the medical field, industrial field, etc. Endoscope use environment is generally harsh, module with a year time basically have problems, and light source with a year time will decay. At present, the light source in the endoscope module on the market needs to be disassembled when replacing the whole machine, resulting in a particularly troublesome endoscope maintenance and repair.

### SUMMARY OF THE DISCLOSURE

Embodiments of the present disclosure provide a camera module and an endoscope.

In a first aspect, a camera module is provided, including:

a connecting base, including a first connecting member and a first circuit board; wherein the first circuit board is installed in the first connecting member; and

a camera, including a housing, a second circuit board, a light source assembly, and a lens assembly; wherein the second circuit board, the light source assembly, and the lens assembly are respectively installed in the housing; the housing is removably connected to the first connecting member; the first circuit board and the second circuit board are electrically connected to each other.

In some embodiments, the second circuit board includes a first board portion, a second board portion, and a third board portion connected in sequence; the first board portion is disposed at an end of the housing facing away from the first connecting member, and the third board portion is disposed on the other end of the housing;

the light source assembly is disposed on a side of the first board portion facing away from the second board portion; the lens assembly is disposed between the first board portion and the second board portion.

In some embodiments, the first board portion is arranged with a heat sink on a side facing away from the light source assembly, the heat sink abutting against the lens assembly; and/or

the second board portion is arranged with a heat sink on a side facing away from the lens assembly, the heat sink abutting against an inner wall of the housing.

In some embodiments, wherein the light source assembly includes a plurality of lamp beads annularly distributed on the first board portion;

the housing defines a light-transmitting window, and the plurality of lamp beads face the light-transmitting window.

In some embodiments, the lens assembly includes an optical lens and an image sensor; the image sensor is disposed at the second board portion; an end of the optical lens is connected to the image sensor;

the first board portion defines a through hole; the other end of the optical lens passes through the through hole and faces the light-transmitting window.

In some embodiments, the housing includes a fixing member and a second connecting member; an end of the second connecting member is sleeved on the fixing member, and the other end of the second connecting member is sleeved on the first connecting member;

an inside of the fixing member defines an installation cavity, and the second circuit board is installed in the installation cavity.

In some embodiments, the fixing member defines a clamping groove, and an end of the second connecting member facing away from the first connecting member is arranged with a clamping portion; the clamping portion is disposed in the clamping groove; an end of the clamping portion facing the first connecting member abuts against a wall of the clamping groove;

the housing further includes a casing; the casing is sleeved on an end of the fixing member facing away from the first connecting member, and the casing abuts against the other end of the clamping portion.

In some embodiments, the camera module further includes a spring pin assembly disposed in the housing and connected between the first circuit board and the second circuit board.

In some embodiments, the spring pin assembly includes a spring pin base and a plurality of spring pins; the plurality of spring pins are installed on the spring pin base;

the first circuit board is arranged with a plurality of metal contacts; the plurality of spring pins abut against the plurality of metal contacts.

In a second aspect, an endoscope is provided, including:

a host; and

a camera module according to any one of the above embodiments; wherein the camera module is further arranged with a signal line connected to the host.

### BRIEF DESCRIPTION OF THE DRAWINGS

One or more embodiments are exemplified by the accompanying drawings. These exemplified descriptions do not constitute a limitation on the embodiments. The elements with the same reference numerals in the drawings are denoted as similar elements, and the figures in the accompanying drawings do not constitute a limitation of scale unless specifically stated.

FIG. 1 is a structural schematic view of a camera module according to an embodiment of the present disclosure.

FIG. 2 is an exploded schematic view of the camera module shown in FIG. 1.

FIG. 3 is a cross-sectional view of the camera module shown in FIG. 1.

FIG. 4 is a cross-sectional view of a first connector of the camera module shown in FIG. 3.

FIG. 5 is a structural schematic view of a first circuit board and a signal line of the camera module shown in FIG. 3.

FIG. 6 is an exploded schematic view of a housing of the camera module shown in FIG. 3.

FIG. 7 is a cross-sectional view of a housing and a first connector of the camera module shown in FIG. 3.

FIG. 8 is a structural schematic view of a second circuit board of the camera module shown in FIG. 3.

FIG. 9 is a structural schematic view of a second circuit board, a spring pin assembly, a light source assembly, and a lens assembly of the camera module shown in FIG. 3.

FIG. 10 is a cross-sectional view of a camera module according to another embodiment of the present disclosure.

FIG. 11 is a structural schematic view of a housing of the camera module shown in FIG. 10.

FIG. 12 is a structural schematic view of a second circuit board, a light source assembly, and a lens assembly of the camera module shown in FIG. 10.

### DETAILED DESCRIPTION

To facilitate the understanding of the present disclosure, the present disclosure will be described in more detail below with reference to the accompanying drawings and specific embodiments. It should be noted that when an element is expressed as being “fixed to” another element, it may be directly on the other element, or there may be one or more elements in between. When an element is expressed as being “connected to” another element, it may be directly connected to the other element, or there may be one or more intervening elements in between. The terms “vertical”, “horizontal”, “left”, “right”, “inner”, “outer” and similar expressions used in this specification are for illustrative purposes only.

Unless otherwise defined, all technical and scientific terms used in this specification have the same meaning as commonly understood by those skilled in the technical field of the present disclosure. The terms used in the description of the present disclosure are only for the purpose of describing specific embodiments and not to limit the present disclosure. The term “and/or” used in this specification includes any and all combinations of one or more related listed items.

In addition, the technical features involved in the different embodiments of the present disclosure described below can be combined with each other as long as they do not conflict with each other.

Referring to FIG. 1, an embodiment of the present disclosure provides a camera module 100, which includes a connecting base 10 and at least one camera 20. The connecting base 10 and the at least one camera 20 are detachably connected. The number of the cameras 20 may be multiple, and the cameras 20 may be respectively configured with different parameters. The cameras 20 may be replaced and installed on the connecting base 10 to change the depth of field, viewing angle, endoscope viewing direction and other parameters of the camera module 100. The camera 20 may be a direct-view camera, a side-view camera, a direct-view camera and a side-view dual camera, etc. In the embodiment, a direct-view camera is taken as an example for description.

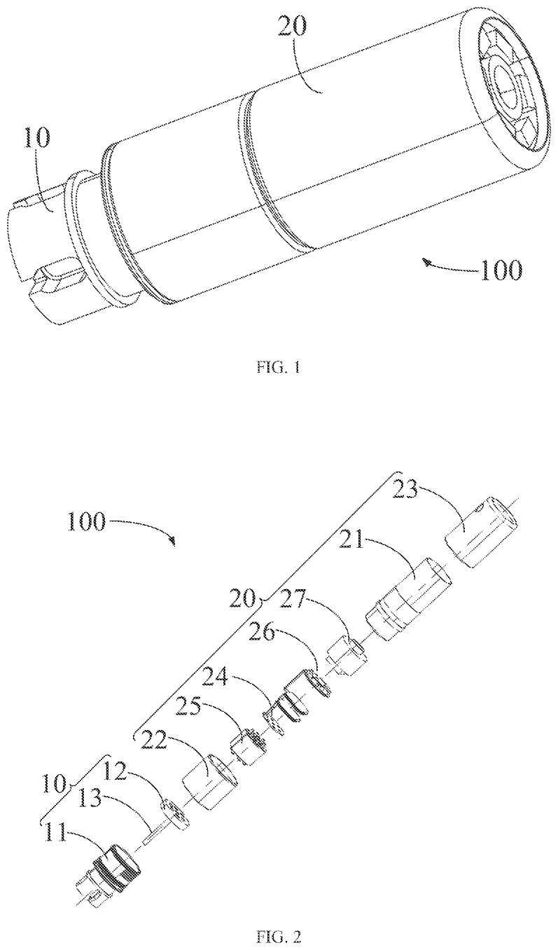

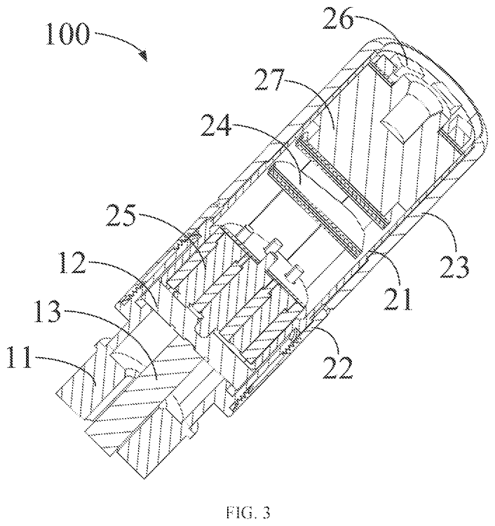

Referring to FIGS. 2 and 3 together, the connecting base 10 may include a first connecting member 11, a first circuit board 12, and a signal line 13. The first circuit board 12 is installed in the first connecting member 11. The signal line 13 is connected to the first circuit board 12. An end of the first connecting member 11 is configured to connect to the camera 20, and the other end of the first connecting member 11 is connected to an external pipe.

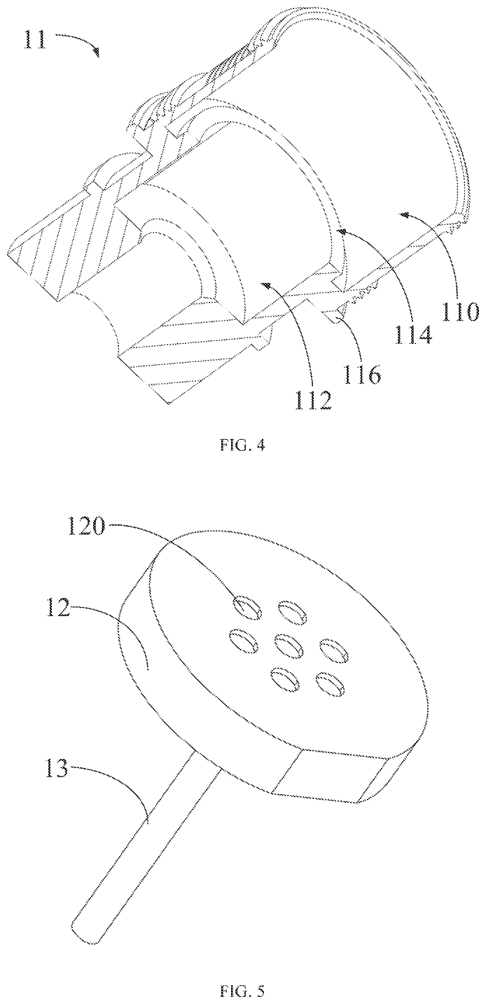

Referring to FIGS. 3 to 5 together, the first connecting member 11 may be a hollow round tube. An inside of the first connecting member 11 defines a stepped hole including a first receiving hole 110 and a second receiving hole 112. The first receiving hole 110 and the second receiving hole 112 are in communication. The diameter of the first receiving hole 110 is greater than the diameter of the second receiving hole 112. A stepped surface 114 is formed between the first receiving hole 110 and the second receiving hole 112. The first receiving hole 110 is disposed facing the camera 20, the first circuit board 12 is disposed in the first receiving hole 110, and the first circuit board 12 abuts the stepped surface 114. The signal line 13 is connected to a side of the first circuit board 12 facing away from the camera 20. The signal line 13 passes through the second receiving hole 112 to be connected to an external host. The first connecting member 11 is arranged with a positioning outer ring 116. The positioning outer ring 116 protrudes from an outer wall of the first connecting member 11. The positioning outer ring 116 is configured to position the camera 20. An end of the first connecting member 11 facing the camera 20 is further arranged with an external thread configured to connect with the camera 20.

It can be understood that in some other embodiments, the shape of the first connecting member 11 may be set according to actual needs, such as a square tube, a triangular tube, an elliptical tube, etc., just to be able to meet the installation of the first circuit board 12 and the signal line 13.

The first circuit board 12 may be plate-shaped. A side of the first circuit board 12 facing away from the camera 20 abuts on the stepped surface 114, and the other side of the first circuit board 12 is arranged with a plurality of metal contacts 120 configured for electrical connection with the camera 20. The shape of the first circuit board 12 matches the shape of the first receiving hole 110, and an outer edge of the first circuit board 12 abuts an inner wall of the first receiving hole 110 to facilitate installation of the first circuit board 12.

Referring to FIGS. 2 and 3 again, the camera 20 may include a housing, a second circuit board 24, a spring pin assembly 25, a light source assembly 26, and a lens assembly 27. The second circuit board 24, the spring pin assembly 25, the light source assembly 26 and the lens assembly 27 are respectively installed in the housing. The housing includes a fixing member 21, a second connecting member 22 and a casing 23. The second connecting member 22 and the casing 23 are respectively connected to the fixing member 21. The spring pin assembly 25 is connected to an end of the second circuit board 24. The light source assembly 26 is connected to the other end of the second circuit board 24. The lens assembly 27 is disposed between the spring pin assembly 25 and the light source assembly 26, and is connected to the second circuit board 24.

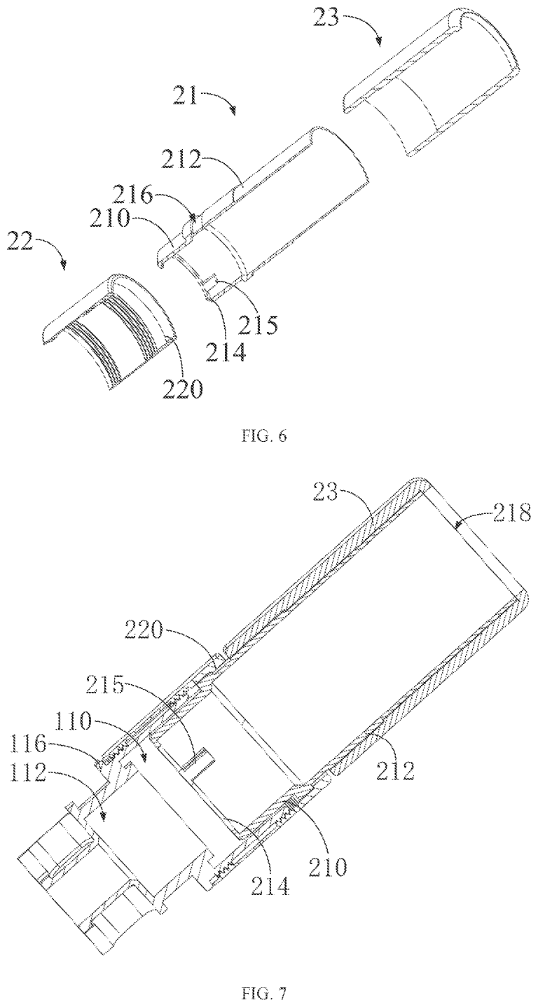

Referring to FIG. 3, FIG. 6, and FIG. 7 together, the fixing member 21 may be a hollow round tube. An inside of the fixing member 21 defines an installation cavity. The second circuit board 24, the spring pin assembly 25, the light source assembly 26 and the lens assembly 27 are respectively installed in the installation cavity. The fixing member 21 may be a stepped shaft and include a first fixing portion 210 and a second fixing portion 212. The first fixing portion 210 and the second fixing portion 212 are connected. The diameter of the first fixing portion 210 is less than the diameter of the second fixing portion 212. The first connecting member 11 is sleeved on the first fixing portion 210. The second connecting member 22 and the casing 23 are sleeved on the second fixing portion 212 respectively. The first fixing portion 210 of the fixing member 21 is disposed in the first receiving hole 110 of the first connecting member 11, and an end of the first fixing portion 210 facing away from the second fixing portion 212 abuts the first circuit board 12.

The end of the first fixing portion 210 facing away from the second fixing portion 212 is further arranged with a positioning inner ring 214. The positioning inner ring 214 protrudes from an inner wall of the first fixing portion 210 toward an inner cavity. The positioning inner ring 210 is configured to position the spring pin assembly 25. The inner wall of the first fixing portion 210 is further arranged with a limiting block 215, the limiting block 215 extending from the positioning inner ring 214 in a direction away from the second fixing portion 212. The limiting block 215 is configured to limit the position of the spring pin assembly 25.

An end of the second fixing portion 212 facing the first fixing portion 210 further defines a clamping groove 216 configured to connect with the second connecting member 22. A groove wall at an end of the clamping groove 216 facing the first fixing portion 212 is an abutting wall configured to limit the position of the second connecting member 22. An end of the second fixing portion 212 facing away from the first fixing portion 210 defines a light-transmitting window 218 communicating the installation cavity with an outside.

The second connecting member 22 may be a hollow round tube, and an end of the second connecting member 22 facing the connecting base 10 is arranged with an internal thread. The second connecting member 22 is sleeved on the first connecting member 11. The internal thread of the second connecting member 22 matches the external thread of the first connecting member 11, such that the second connecting member 22 is fixed to the first connecting member 11. An end of the second connecting member 22 facing away from the connecting base 10 is arranged with a clamping portion 220, and the clamping portion 220 protrudes from an inner wall of the second connecting member 22. An end of the second connecting member 22 facing away from the connecting base 10 is sleeved on the second fixing portion 212 of the fixing member 21. The clamping portion 220 is disposed in the clamping groove 216. An end of the clamping portion 220 facing the first connecting member 11 abuts against the abutting wall.

It can be understood that, in some other embodiments, the connection mode of the second connecting member 22 and the first connecting member 11 may be set according to actual needs, such as a snap connection, a fastener connection, a key connection, etc., just to be able to meet the detachable connection requirement between the first connecting member 11 and the second connecting member 22.

The casing 23 may be a hollow round tube. The casing 23 is sleeved on an end of the second fixing portion 212 facing away from the first fixing portion 210. The casing 23 abuts against an end of the clamping portion 220 of the second connecting member 22 away from the first connecting member 11, such that the second connecting member 22 is axially fixed. A window corresponding to the light-transmitting window 218 is defined at an end of the casing 23 facing away from the second connecting member 22 to ensure that the light-transmitting window 218 communicates with the outside.

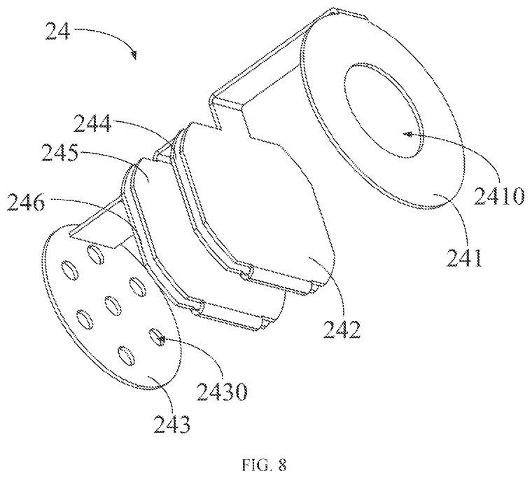

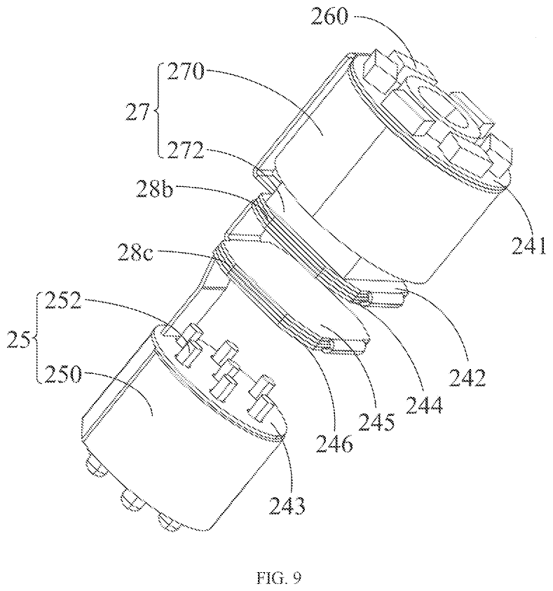

Referring to FIGS. 8 and 9 together, the second circuit board 24 may be a flexible circuit board, and the second circuit board 24 may include a first board portion 241, a second board portion 242, and a third board portion 243 connected in sequence. The first board portion 241, the second board portion 242, and the third board portion 243 are sequentially arranged along an axial direction of the fixing member 21. The first board portion 241, the second board portion 242 and the third board portion 243 are spaced apart. The first board portion 241 is disposed at an end of the second fixing portion 212 facing away from the first fixing portion 210, and the third board portion 243 is disposed on the other end of the second fixing portion 212.

The first board portion 241 may have an annular plate shape and define a through hole 2410. The light source assembly 26 is arranged on a side of the first board portion 241 facing away from the second board portion 242 and faces the light-transmitting window 218. The emitted light can be transmitted through the light-transmitting window 218 to the outside. The lens assembly 27 is disposed between the first board portion 241 and the second board portion 242, and two ends of the lens assembly 27 are respectively connected to the first board portion 241 and the second board portion 242. An end of the lens assembly 27 passes through the through hole 2410. The third board portion 243 defines a plurality of connecting holes 2430 configured to connect with the spring pin assembly 25.

In some embodiments, the first board portion 241 is arranged with a heat sink 28a on a side facing away from the light source assembly 26, the heat sink 28a abuts against the lens assembly 27, and the heat sink 28a abuts against an inner wall of the fixing member 21. The heat sink 28a can conduct the heat generated during the operation of the light source assembly 26 and the lens assembly 27 to the housing. The second board portion 242 is further arranged with a heat sink 28b on a side facing away from the lens assembly 27, the heat sink 28b abuts against the inner wall of the fixing member 21, and the heat sink 28b can conduct the heat generated during the operation of the lens assembly 27 to the housing.

In some embodiments, the second circuit board 24 may further include a fourth board portion 244, a fifth board portion 245, and a sixth board portion 246 that are sequentially connected. The fourth board portion 244, the fifth board portion 245, and the sixth board portion 246 are sequentially arranged at intervals along the axial direction of the fixing member 21. The fourth board portion 244, the fifth board portion 245, and the sixth board portion 246 are disposed between the second board portion 242 and the third board portion 243. The fourth board portion 244 is connected to the second board portion 242, and the sixth board portion 246 is connected to the third board portion 243. The heat sink 28b is disposed between the second board portion 242 and the fourth board portion 244. A heat sink 28c is further arranged between the fifth board portion 245 and the sixth board portion 246. The heat sink 28c abuts against the inner wall of the fixing member 21 for further dissipating heat for the light source assembly 26 and the lens assembly 27.

In some embodiments, the installation cavity of the fixing member 21 is potted with thermally conductive glue, such that the second circuit board 24 and the fixing member 21 are fixedly connected, and the heat conduction between the second circuit board 24 and the fixing member 21 is increased, which can better dissipate heat for the light source assembly 26 and the lens assembly 27.

The spring pin assembly 25 may include a spring pin base 250 and a plurality of spring pins 252. The plurality of spring pins 252 are installed on the spring pin base 250. A plurality of installation through holes (not shown) are defined on the spring pin base 250. The installation through holes penetrate through two ends of the spring pin base 250 in an axial direction. Each spring pin 252 is installed in a corresponding installation through hole.

The spring pin base 250 is disposed in the first fixing portion 210. An end of the spring pin base 250 abuts the positioning inner ring 214, and the other end of the spring pin base 250 abuts the third board portion 243, such that the spring pin base 250 is fixed along the axial direction of the fixing member 21. An outer wall of the spring pin base 250 defines a limiting groove 2500. The shape of the limiting groove 2500 matches the shape of the limiting block 215, and the limiting block 215 is clamped in the limiting slot 2500, such that the spring pin base 250 is fixed along the axial direction of the fixing member 21.

An end of each spring pin 252 is disposed in one of the plurality of connecting holes 2430 of the third board portion 234, and each spring pin 252 corresponds to one of the connecting holes 2430. The plurality of spring pins 252 are welded and fixed to the third board portion 234. The other end of each spring pin 252 abuts against the plurality of metal contacts 120 of the first circuit board 12, and each spring pin 252 corresponds to one of the metal contacts 120. The spring pins 252 can enable the electrical connection between the first circuit board 12 and the second circuit board 23, and can conduct heat on the second circuit board 23 to the first circuit board 12 for heat dissipation.

The light source assembly 26 may include a plurality of lamp beads 260 annularly distributed on the first board portion 241. The plurality of lamp beads 260 are welded to the first board portion 241 such that the plurality of lamp beads 260 are electrically connected to the second circuit board 24. Light-emitting ends of the plurality of lamp beads 260 face the light-transmitting window 218, and the plurality of lamp beads 260 can emit light toward the outside of the camera 20. In the embodiments, each lamp bead may be a light-emitting diode (LED), and the LED can have any light source wavelength band to meet different requirements.

The lens assembly 27 may include an optical lens 270 and an image sensor 272. An end of the optical lens 270 is connected to the image sensor 272. The other end of the optical lens 270 passes through the through hole 2410 to communicate with the outside. The optical lens 270 is configured to refract and filter light. The image sensor 272 is soldered to the second board portion 242 such that the image sensor 272 is electrically connected to the second circuit board 24. The external light enters through the light-transmitting window 218 and is refracted by the optical lens 270 and transmitted to the image sensor 272. The image sensor 272 generates image information after processing.

The assembly process of the camera module 100 will be briefly described below in conjunction with the drawings.

When the connecting base 10 is assembled, the first circuit board 12 and the signal line 13 are welded first, the signal line 13 passes through the first receiving hole 110 and the second receiving hole 112 in sequence, and the first circuit board 12 is disposed in the first receiving hole 110 and abuts the stepped surface 114.

When the camera 20 is assembled, the second circuit board 24 is bent, and the spring pin assembly 25, the light source assembly 26, and the lens assembly 27 are respectively welded on the second circuit board 24. The second circuit board 24, the spring pin assembly 25, the light source assembly 26, and the lens assembly 27 are installed in the fixing member 21 after assembled. The second connecting member 22 is sleeved on the fixing member 21, and the casing 23 is sleeved on the fixing member 21 and abuts against the second connecting member 22.

When the connecting base 10 is assembled with the camera 20, the second connecting member 22 is sleeved on the first connecting member 11, the second connecting member 22 is rotated to screw with the first connecting member 11 until the second connecting member 22 abuts against the positioning outer ring 116, and the spring pins 252 abut against the metal contacts 120.

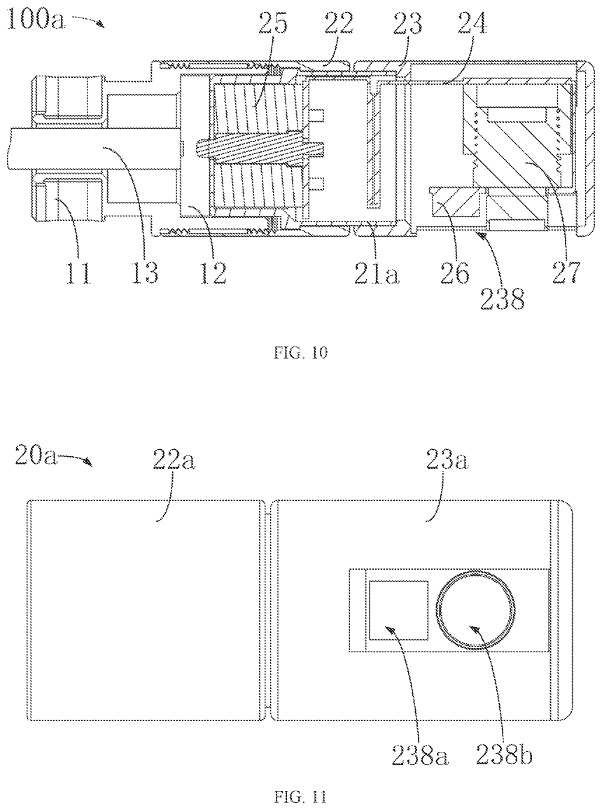

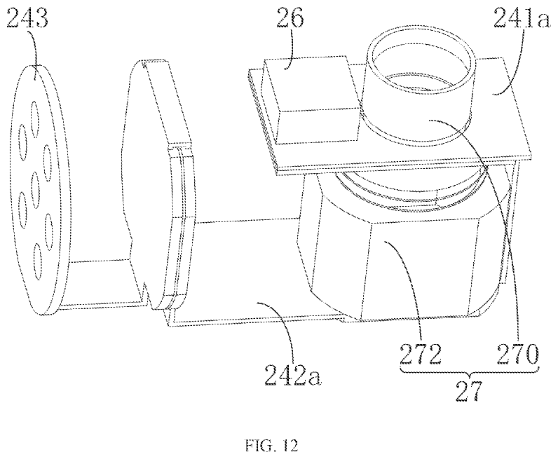

Referring to FIGS. 10 to 12, another embodiment of the present disclosure provides a camera module 100a which is basically the same as the camera module 100 in the above embodiments, except that the camera 20a of the camera module 100a is a side-view camera. Specifically, the end of the casing 23a facing away from the second connecting member 22a extends axially out of the fixing member 21a, and the inner cavity of the casing 23a is in communication with the installation cavity of the fixing member 21a. The light-transmitting window 238 is defined on a side wall of the end of the casing 23a facing away from the second connecting member 22a. The light-transmitting window 238 communicates the inner cavity of the casing 23a with the outside, and the end of the casing 23a away from the second connecting member 22a is not in communication with the outside. The light-transmitting window 238 includes a first light-transmitting window 238a and a second light-transmitting window 238b distributed along the axial direction of the casing 23a. The second circuit board 24a extends from the installation cavity to the inner cavity of the casing 23a, the first board portion 241a and the second board portion 242a are disposed in the inner cavity of the casing 23a, and the first board portion 241a and the second board portion 242a are arranged along a radial direction of the casing 23a. The light source assembly 26 is arranged on the side of the first board portion 241a facing the light-transmitting window 238, and the position of the light source assembly 26 corresponds to the position of the first light-transmitting window 238a. The lens assembly 27 is disposed between the first board portion 241a and the second board portion 242a, the optical lens 270 is disposed on a side of the light source assembly 26 along the axial direction of the casing 23a, and the position of the optical lens 270 corresponds to the position of the second light-transmitting window 238b. Based on the above method, the camera module 100a provided in this embodiment can realize lateral shooting and meet the shooting requirements in different environments.
1. Another embodiment of the present disclosure also provides a camera module 100b which is basically the same as the camera module 100 in the foregoing embodiment, except that the camera of the camera module 100b is a direct-view and a side-view dual camera. Specifically, the end of the second fixing portion away from the first fixing portion defines a light-transmitting window, and the side wall of the second fixing portion defines another light-transmitting window. The casing defines two windows corresponding to the two light-transmitting windows, such that the two light-transmitting windows are respectively connected to the outside. The second circuit board is arranged with two light source assemblies and two lens assemblies, each light source assembly faces a corresponding one of the light-transmitting windows, and each lens assembly faces a corresponding one of the light-transmitting windows. Based on the above method, the camera module 100a provided in this embodiment can realize simultaneous vertical and lateral shooting, which meets the shooting requirements in different environments.
2. Another embodiment of the present disclosure provides an endoscope. The endoscope includes a host and the camera module described in any of the above embodiments. The signal line of the camera module is connected to the host to transmit image information of the image sensor to the host.

Finally, it should be noted that the above embodiments are only to illustrate the technical solutions of the present disclosure, not to limit them; under the idea of the present disclosure, the technical features of the above embodiments or different embodiments can also be combined. The steps can be implemented in any order, and there are many other variations of the different aspects of the present disclosure as described above. For the sake of brevity, they are not provided in details; although the present disclosure has been described in detail with reference to the foregoing embodiments, those skilled in the art should understand that: they can still modify the technical solutions recorded in the foregoing embodiments, or equivalently replace some of the technical features; and these modifications or replacements do not make the essence of the corresponding technical solutions out of the scope of the technical solutions of the various embodiments of the present disclosure.

What is claimed is:

1. A camera module, comprising:
a connecting base, comprising a first connecting member and a first circuit board; wherein the first circuit board is installed in the first connecting member; and
a camera, comprising a housing, a second circuit board, a light source assembly, and a lens assembly; wherein the second circuit board, the light source assembly, and the lens assembly are respectively installed in the housing; the housing is removably connected to the first connecting member; the first circuit board and the second circuit board are electrically connected to each other.

2. The camera module according to claim 1, wherein the second circuit board comprises a first board portion, a second board portion, and a third board portion connected in sequence; the first board portion is disposed at an end of the housing facing away from the first connecting member, and the third board portion is disposed on the other end of the housing;
the light source assembly is disposed on a side of the first board portion facing away from the second board portion; the lens assembly is disposed between the first board portion and the second board portion.

3. The camera module according to claim 2, wherein the first board portion is arranged with a heat sink on a side facing away from the light source assembly, the heat sink abutting against the lens assembly; and/or
the second board portion is arranged with a heat sink on a side facing away from the lens assembly, the heat sink abutting against an inner wall of the housing.

4. The camera module according to claim 2, wherein the light source assembly comprises a plurality of lamp beads annularly distributed on the first board portion;
the housing defines a light-transmitting window, and the plurality of lamp beads face the light-transmitting window.

5. The camera module according to claim 4, wherein the lens assembly comprises an optical lens and an image sensor; the image sensor is disposed at the second board portion; an end of the optical lens is connected to the image sensor;
the first board portion defines a through hole; the other end of the optical lens passes through the through hole and faces the light-transmitting window.

6. The camera module according to claim 1, wherein the housing comprises a fixing member and a second connecting member; an end of the second connecting member is sleeved on the fixing member, and the other end of the second connecting member is sleeved on the first connecting member;
an inside of the fixing member defines an installation cavity, and the second circuit board is installed in the installation cavity.

7. The camera module according to claim 6, wherein the fixing member defines a clamping groove, and an end of the second connecting member facing away from the first connecting member is arranged with a clamping portion; the clamping portion is disposed in the clamping groove; an end of the clamping portion facing the first connecting member abuts against a wall of the clamping groove;
the housing further comprises a casing; the casing is sleeved on an end of the fixing member facing away from the first connecting member, and the casing abuts against the other end of the clamping portion.

8. The camera module according to claim 1, further comprising a spring pin assembly disposed in the housing and connected between the first circuit board and the second circuit board.

9. The camera module according to claim 8, wherein the spring pin assembly comprises a spring pin base and a plurality of spring pins; the plurality of spring pins are installed on the spring pin base;
the first circuit board is arranged with a plurality of metal contacts; the plurality of spring pins abut against the plurality of metal contacts.

10. An endoscope, comprising:
a host; and
a camera module, comprising:
a connecting base, comprising a first connecting member and a first circuit board; wherein the first circuit board is installed in the first connecting member; and
a camera, comprising a housing, a second circuit board, a light source assembly, and a lens assembly; wherein the second circuit board, the light source assembly, and the lens assembly are respectively installed in the housing; the housing is removably connected to the first connecting member; the first circuit board and the second circuit board are electrically connected to each other;
wherein the camera module is further arranged with a signal line connected to the host.

11. The endoscope according to claim 10, wherein the second circuit board comprises a first board portion, a second board portion, and a third board portion connected in sequence; the first board portion is disposed at an end of the housing facing away from the first fixing portion, and the third board portion is disposed on the other end of the housing;
the light source assembly is disposed on a side of the first board portion facing away from the second board portion; the lens assembly is disposed between the first board portion and the second board portion.

12. The endoscope according to claim 11, wherein the first board portion is arranged with a heat sink on a side facing away from the light source assembly, the heat sink abutting against the lens assembly; and/or
the second board portion is arranged with a heat sink on a side facing away from the lens assembly, the heat sink abutting against an inner wall of the housing.

13. The endoscope according to claim 11, wherein the light source assembly comprises a plurality of lamp beads annularly distributed on the first board portion;
the housing defines a light-transmitting window, and the plurality of lamp beads face the light-transmitting window.

14. The endoscope according to claim 13, wherein the lens assembly comprises an optical lens and an image sensor; the image sensor is disposed at the second board portion; an end of the optical lens is connected to the image sensor;
the first board portion defines a through hole; the other end of the optical lens passes through the through hole and faces the light-transmitting window.

15. The endoscope according to claim 10, wherein the housing comprises a fixing member and a second connecting member; an end of the second connecting member is sleeved on the fixing member, and the other end of the second connecting member is sleeved on the first connecting member;
an inside of the fixing member defines an installation cavity, and the second circuit board is installed in the installation cavity.

16. The endoscope according to claim 15, wherein the fixing member defines a clamping groove, and an end of the second connecting member facing away from the first connecting member is arranged with a clamping portion; the clamping portion is disposed in the clamping groove; an end of the clamping portion facing the first connecting member abuts against a wall of the clamping groove;
the housing further comprises a casing; the casing is sleeved on an end of the fixing member facing away from the first connecting member, and the casing abuts against the other end of the clamping portion.

17. The endoscope according to claim 10, wherein the camera module further comprises a spring pin assembly disposed in the housing and connected between the first circuit board and the second circuit board.

18. The endoscope according to claim 17, wherein the spring pin assembly comprises a spring pin base and a plurality of spring pins; the plurality of spring pins are installed on the spring pin base;
the first circuit board is arranged with a plurality of metal contacts; the plurality of spring pins abut against the plurality of metal contacts.
